SEQUENCE LISTING (14 sequences)

SEQ ID NO: 1 - DNA, 780 bp, Zoogloea ramigera
atgtcgttgaatggcaaagtcattttggtaaccggcgctggccaggggatcggacgcggtattgcgctgcggcttgcaaaggaaggcgctgatctcgcgctggccgacgtcaaggccgataagctcgactccgttcgcaaggaagtcgaagcgctcggacgcaaggccaccaccgtcgtcgccgatgtcagcaagcgcgacgaagtctacgccgccatcgaccatgcagagaagcaactcggcgggttcgacgtcatggtcaacaatgccggcatcgcccaggtcaagccgatcgccgacgtcacgcccgaggacatggacctgatcttccggatcaatgtcgacggcgtgctctggggcatccaggcggcttcgcagaaattcaaggatcgaggtcagaagggcaagatcatcaatgcctgctcgattgccggacatgacggcttcgccatgctcggcgtctattcggcaaccaagttcgccgtgcgtgcgctgacgcaagccgccgccaaggaatatgccagcgcgggcatcacggtgaacgcctactgccccggcatcgtcggcaccgacatgtgggtggagatcgacgagcgcttttccgagatcaccggaacgccgaagggcgaaacctacaagaaatacgtcgagggcatcgctttgggccgcgcgcagacaccggaggacgtggcagcgttggtcgccttcctcgcgggcgccgattccgactacatcacgggacagtcgatcctgaccgatggcggtatcgtctatcgataa

SEQ ID NO: 2 - protein, 259 aa, Zoogloea ramigera
MSLNGKVILVTGAGQGIGRGIALRLAKEGADLALADVKADKLDSVRKEVEALGRKATTVVADVSKRDEVYAAIDHAEKQLGGFDVMVNNAGIAQVKPIADVTPEDMDLIFRINVDGVLWGIQAASQKFKDRGQKGKIINACSIAGHDGFAMLGVYSATKFAVRALTQAAAKEYASAGITVNAYCPGIVGTDMWVEIDERFSEITGTPKGETYKKYVEGIALGRAQTPEDVAALVAFLAGADSDYITGQSILTDGGIVYR

SEQ ID NO: 3 - protein, 11 aa, Zoogloea ramigera
MSLNGKVILVT

SEQ ID NO: 4 - protein, 10 aa, Zoogloea ramigera
IINACSIAGH

SEQ ID NO: 5 - DNA, 32 bp, Artificial
  an artificially synthesized primer sequence
gtcgaattcaayggcaargtsatyytsgtnac

SEQ ID NO: 6 - DNA, 31 bp, Artificial
  an artificially synthesized primer sequence
gtcgaattcgcratsgarcasgcrttratda

SEQ ID NO: 7 - DNA, 31 bp, Artificial
  an artificially synthesized primer sequence
gtcgaattcgcratrctrcasgcrttratda

SEQ ID NO: 8 - DNA, 380 bp, Zoogloea ramigera
accggcgctggccaggggatcggacgcggtattgcgctgcggcttgcaaaggaaggcgctgatctcgcgctggccgacgtcaaggccgataagctcgactccgttcgcaaggaagtcgaagcgctcggacgcaaggccaccaccgtcgtcgccgatgtcagcaagcgcgacgaagtctacgccgccatcgaccatgcagagaagcaactcggcgggttcgacgtcatggtcaacaatgccggcatcgcccaggtcaagccgatcgccgacgtcacgcccgaggacatggacctgatcttccggatcaatgtcgacggcgtgctctggggcatccaggcggcttcgcagaaattcaaggatcgaggtcagaagggcaagat

SEQ ID NO: 9 - DNA, 18 bp, Artificial
  an artificially synthesized primer sequence
gcgagatcagcgccttcc

SEQ ID NO: 10 - DNA, 18 bp, Artificial
  an artificially synthesized primer sequence
tgtcgacggcgtgctctg

SEQ ID NO: 11 - DNA, 451 bp, Zoogloea ramigera
cgaaccgccgatccagcgccgctccatgacggctaccttctggccggcttgcgccatatcccaggcgaggtatttgccgccctcgccactgccgatcacgacagcgtcgtagcgctccgcttccgtcatcaactacgtatccttctgcaacagcgactcaccgcacgatgaaaatatggcgctcggacatatatgcggaatcatgtcatactcttgcggggtcgaacaattactgttgcttttgcgggcggccccttgaggtggccttactgccagtatccgtgccgtcgcatcatttccaggccacggccttaaaatgggccgggatcatggagtggagtgggttctgctccggctgtattgcccggaaagggaacatgctggctgcacaggattggccgactttctgtaggagaaaatcatgtcgttgaatggcaaagtcattttggta

SEQ ID NO: 12 - DNA, 482 bp, Zoogloea ramigera
catcaatgcctgctcgattgccggacatgacggcttcgccatgctcggcgtctattcggcaaccaagttcgccgtgcgtgcgctgacgcaagccgccgccaaggaatatgccagcgcgggcatcacggtgaacgcctactgccccggcatcgtcggcaccgacatgtgggtggagatcgacgagcgcttttccgagatcaccggaacgccgaagggcgaaacctacaagaaatacgtcgagggcatcgctttgggccgcgcgcagacaccggaggacgtggcagcgttggtcgccttcctcgcgggcgccgattccgactacatcacgggacagtcgatcctgaccgatggcggtatcgtctatcgataaagccgcctgccaaatcgcgaacgttcagaagcgcccacaccgggaaacggccttgtgagaatttttctctccagcggaatggttccgtctggaagaaccgcgttttgtcgga

SEQ ID NO: 13 - DNA, 46 bp, Artificial
  an artificially synthesized primer sequence
gtcgaattcaatcatgagtttaaatggcaaagtcattttggtaacc

SEQ ID NO: 14 - DNA, 38 bp, Artificial
  an artificially synthesized primer sequence
gtcaagcttctagattaacgatagacgataccgccatc

(19) United States
(12) Patent Application Publication
(10) Pub. No.: US 2005/0003500 A1
(43) Pub. Date: Jan. 6, 2005

(54) (2S, 3S) -2,3-butanediol dehydrogenase

(75) Inventors: Masatake Kudoh, Ibaraki (JP); Hiroaki Yamamoto, Ibaraki (JP)

Correspondence Address: LAHIVE & COCKFIELD, LLP., 28 STATE STREET, BOSTON, MA, 02109, US

(73) Assignee: Daicel Chemical Industries, Ltd., Osaka (JP)

(21) Appl. No.: 10/858,707

(22) Filed: Jun. 2, 2004

(30) Foreign Application Priority Data
Jun. 6, 2003 (JP) 2003-162872

(51) Int. Cl. C12P 7/18

(52) U.S. Cl. 435/158

(57) ABSTRACT

An objective of the present invention is to produce a novel (2S,3S)-2,3-butanediol dehydrogenase useful for the production of ketones, alcohols, and particularly optically active vicinal diols. Zoogloea ramigera has been found to produce a novel (2S,3S)-2,3-butanediol dehydrogenase having high activity and high stereoselectivity. Furthermore, a DNA chain encoding this (2S,3S)-2,3-butanediol dehydrogenase was cloned and the nucleotide sequence thereof was determined. The expression of the (2S,3S)-2,3-butanediol dehydrogenase was carried out in a heterologous microorganism.

### FIELD OF THE INVENTION

The present invention relates to a novel nicotinamide adenine dinucleotide-dependent (2S,3S)-2,3-butanediol dehydrogenase. The present invention also relates to polynucleotides encoding the enzyme protein, a method for producing the enzyme and methods for producing alcohols, particularly (2S,3S)-2,3-butanediol and (S)-2-butanol, using the enzyme.

### BACKGROUND OF THE INVENTION

(2S,3S)-2,3-butanediol dehydrogenase is an enzyme which plays important roles in the fermentation production of (2S,3S)-2,3-butanediol with microorganisms using glucose as raw material and in 2,3-butanediol metabolism in microorganisms. Furthermore, (2S,3S)-2,3-butanediol generated via the enzyme reaction is a useful compound as a raw material for the synthesis of liquid crystals, pharmaceutical agents, etc. Moreover, (S)-2-butanol produced by this enzymatic reaction is a compound that is useful as a raw material for flavors, pharmaceutical agents, and so on.

(2S,3S)-2,3-butanediol dehydrogenase is a dehydrogenase that has the activity to preferentially oxidize (2S, 3S)-2,3-butanediol among three isomers of 2,3-butanediol.

Previously, based on studies concerning biosynthesis and metabolism of 2,3-butanediol, regarding enzymes having the activity of 2,3-butanediol dehydrogenation, dehydrogenase activity toward (2S,3S)-2,3-butanediol has been reported to be contained, for example, in the microorganisms listed below (Arch. Microbiol. 116, 197-203, 1978; J. Ferment. Technol. 61, 467-471 ,1983). However, assays for the activity in such previous studies were conducted using only cell-free extract, and thus a variety of properties such as stereoselectivity and specific activity of 2,3-butanediol dehydrogenase remained unclear due to the coexistence of various enzymes.
Serratia marcescens
Staphylococcus aureus
Enterobacter aerogenes
Erwinia carotovora
Brevibacterium saccharolyticum C-1012
Brevibacterium ammoniagenes IAM1641

With respect to enzymes that have been highly purified and had their various properties clarified, the following enzymes have been shown to have the activity of 2,3-butanediol dehydrogenase. However, only their activities for DL-form are known and there is no report on their stereoselectivity. Furthermore, the activities of these 2,3-butanediol dehydrogenases, with the exception of that of Pichia ofunaensis, are comparable to or lower than the activity of glycerol dehydrogenase and, thus, their specific activities are generally low.

Glycerol dehydrogenase derived from Achromobacter liquidum (Achromobacter liquidum KY 3047) (Examined Published Japanese Patent Application No. (JP-B) Sho 58-40467);

Glycerol dehydrogenase derived from Bacillus sp. (Bacillus sp. G-1) (JP-B Hei 03-72272);

Glycerol dehydrogenase derived from Bacillus stearothermophilus (Biochim. Biophys. Acta 994, 270-279 (1989));

Glycerol dehydrogenase derived from Citrobacter freundii (Citrobacter freundii DSM 30040) (J. Bacteriol. 177, 4392-4401 (1995));

Glycerol dehydrogenase derived from Erwinia aroideae(Erwinia aroideae IFO 3830 (Chem. Pharm. Bull. 26, 716-721 (1978));

Glycerol dehydrogenase derived from Geotrichum candidum (Geotrichum candidum IFO 4597 (JP-B Hei 01-27715);

Dihydroxyacetone reductase derived from Pichia ofunaensis (Pichia ofunaensis AKU 4328 (J. Biosci. Bioeng. 88, 148-152 (1999)); and

Glycerol dehydrogenase derived from Schizosaccharomyces pombe (J. Gen. Microbiol. 131, 1581-1588 (1985)).

L-2,3-butanediol dehydrogenase produced from Brevibacterium saccharolyticum (Brevibacterium saccharolyticum C-1012) is known as a highly purified enzyme that has been shown to have a high selectivity towards the (2S,3S)-form of 2,3-butanediol (Biosci. Biotechnol. Biochem. 65, 1876-1878 (2001)). However, this enzyme lacks activity towards 2-butanol, and its selectivity towards optically active secondary alcohols has not been reported (Biosci. Biotechnol. Biochem. 65(8), 1876-1878 (2001)).

In addition, a gene encoding a 2,3-butanediol dehydrogenase participating in the metabolism of 2,3-butanediol has been cloned from Pseudomonas putida and expressed in E. coli (FEMS Microbiol. Lett. 124 (2), 141-150 (1994)); however, the stereoselectivity of this enzyme has not yet been reported. Furthermore, genomic analysis of Pseudomonas aeruginosa has identified a gene that has high homology to the 2,3-butanediol dehydrogenase gene derived from Pseudomonas putida. However, this gene has not yet been recombinantly expressed and, thus, neither its enzyme activity nor stereoselectivity has been verified.

It is an industrially important goal to discover a (2S,3S)-2,3-butanediol dehydrogenase having high stereoselectivity and high specific activity and capable of producing optically active alcohols, such as (2S,3S)-2,3-butanediol and (S)-2-butanol; and particularly, to isolate a gene encoding such an enzyme and prepare transformants capable of expressing the enzyme to enable the ready production of the enzyme on a large scale.

### SUMMARY OF THE INVENTION

An objective of the present invention is to provide a (2S,3S)-2,3-butanediol dehydrogenase that can use NAD⁺ as a coenzyme. Another objective of the present invention is to provide a (2S,3S)-2,3-butanediol dehydrogenase capable of yielding products having high optical purity in high yield when utilized in an enzymatic production process of optically active (2S,3S)-2,3-butanediol using 2,3-butanedione as a substrate. In addition, another objective of the present invention is to provide a (2S,3S)-2,3-butanediol dehydrogenase capable of providing products having high optical purity in high yield when utilized in an enzymatic production process of optically active (S)-2-butanol using 2-butanone as a substrate.

Yet another objective of the present invention is to isolate polynucleotides encoding such a (2S,3S)-2,3-butanediol dehydrogenase having the above-noted desired properties and obtain recombinants thereof. In addition, still another objective is to provide methods for enzymatically producing optically active (2S,3S)-2,3-butanediol and (S)-2-butanol using the novel (2S,3S)-2,3-butanediol dehydrogenase.

The present inventors have performed research relating to Zoogloea ramigera. As a result, a novel (2S,3S)-2,3-butanediol dehydrogenase that is both highly active and, at the same time, highly selective towards the (S)-configuration hydroxyl groups of 2,3-butanediol was discovered.

Furthermore, the present invention was completed by isolating the DNA encoding the enzyme and producing recombinant bacteria that overexpress the enzyme.

Specifically, the present invention relates to following (2S,3S)-2,3-butanediol dehydrogenase, polynucleotides encoding the enzyme, method of producing the enzyme and uses thereof:
[1] a (2S,3S)-2,3-butanediol dehydrogenase having the physicochemical properties of (1) to (3):
(1) Function:
   produces (S)-acetoinbyactingon (2S,3S)-2,3-butanediol using nicotinamide adenine dinucleotide as a coenzyme; and reduces 2,3-butanedione using the reduced form of nicotinamide adenine dinucleotide as a coenzyme to produce (2S,3S)-2,3-butanediol.
(2) Substrate specificity:
   uses nicotinamide adenine dinucleotide as a coenzyme for oxidation reaction; utilizes the reduced form of nicotinamide adenine dinucleotide as a coenzyme for reduction reaction; and preferentially oxidizes (2S,3S)-2,3-butanediol among the three isomers of 2,3-butanediol.
(3) Activation by divalent ions:
   substantially, not activated by Mg²⁺, Ca²⁺, Ba²⁺, Co²⁺, or Mn²⁺ ion;
[2] the (2S,3S)-2,3-butanediol dehydrogenase of [1], which further has the following substrate specificity:
(i) preferentially oxidizes the hydroxyl group in the (S)-configuration of 2-butanol;
(ii) preferentially oxidizes the hydroxyl groups in the (S)-configuration of 1,2-propanediol;
[3] the (2S,3S)-2,3-butanediol dehydrogenase of [1] produced by a microorganism belonging to the genus Zoogloea;
[4] the (2S,3S)-2,3-butanediol dehydrogenase of [3], wherein the microorganism belonging to the genus Zoogloea is Zoogloea ramigera;
[5] a polynucleotide selected from the group of (a) to (e):
(a) a polynucleotide comprising the nucleotide sequence of SEQ ID NO: 1;
(b) a polynucleotide encoding a protein consisting essentially of the amino acid sequence of SEQ ID NO: 2;
(c) a polynucleotide encoding a protein consisting of the amino acid sequence of SEQ ID NO: 2 wherein one or more amino acids have been substituted, deleted, inserted and/or added, which protein is functionally equivalent to the protein consisting essentially of the amino acid sequence of SEQ ID NO: 2;
(d) a polynucleotide that hybridizes under stringent conditions with a DNA consisting essentially of the nucleotide sequence of. SEQ ID NO: 1, and which encodes a protein functionally equivalent to the protein consisting essentially of the amino acid sequence of SEQ ID NO: 2; and
(e) a polynucleotide comprising a nucleotide sequence that has 70% or higher homology to the nucleotide sequence of SEQ ID NO: 1;
[6] a protein encoded by the polynucleotide of [5];
[7] a vector comprising the polynucleotide of [5];
[8] a transformant carrying the polynucleotide of [5] or the vector of [7];
[9] a method of producing the enzyme of [1] or the protein of [6], wherein the method comprises the step of culturing a microorganism that belongs to the genus Zoogloea and that produces the enzyme of [1] or the protein of [6];
[10] the method of [9], wherein the microorganism belonging to the genus Zoogloea is Zoogloea ramigera;
[11] a method of producing the enzyme of [1] or the protein of [6], wherein the method comprises the steps of culturing the transformant of [8] and collecting the expression product;
[12] a method of producing an alcohol, wherein the method comprises the steps of: (1) reacting an enzymatically active substance selected from the group consisting of the (2S,3S)-2,3-butanediol dehydrogenase of [1], the protein of [6], microorganisms producing them and treated products thereof, with a ketone in the presence of the reduced form of nicotinamide adenine dinucleotide; and (2) collecting the produced alcohol;
[13] the method of producing an alcohol of [12], wherein the ketone is 2,3-butanedione and the alcohol is (2S,3S)-2,3-butanediol; and,
[14] a method of producing an optically active alcohol, wherein the method comprises the steps of: (1) reacting an enzymatically active substances selected from the group consisting of the (2S,3S)-2,3-butanediol dehydrogenase of [1], the protein of [6], microorganisms producing them and treated products thereof, with a racemic alcohol in the presence of the reduced form of nicotinamide adenine dinucleotide; (2) preferentially oxidizing one of the optical isomers; and (3) obtaining the remaining optically active alcohol.

It is to be understood that both the foregoing summary of the invention and the following detailed description are of a preferred embodiment, and not restrictive of the invention or other alternate embodiments of the invention.

### BRIEF DESCRIPTION OF THE DRAWINGS

FIG. 1 is a photograph depicting the result of SDS-PAGE analysis on concentrated fractions that exhibited (2S,3S)-2,3-butanediol dehydrogenase activity.

FIG. 2 depicts the results of measuring the optimal pH for oxidation reaction by (2S,3S)-2,3-butanediol dehydrogenase. The results are expressed as relative activity wherein the maximum activity is taken as 100.

FIG. 3 depicts the results of measuring the optimal pH for reduction reaction by (2S,3S)-2,3-butanediol dehydrogenase. The results are expressed as relative activity where the maximum activity is taken das 100.

FIG. 4 depicts the results of measuring the optimal temperature for (2S,3S)-2,3-butanediol dehydrogenase reaction. The results are expressed as relative activity where the maximum activity is taken as 100.

FIG. 5 is a plasmid map of the pSE-ZRD1 plasmid obtained in Example 14.

FIG. 6 is a plasmid map of the pSF-ZRD1 plasmid obtained in Example 17.

### DETAILED DESCRIPTION OF THE INVENTION

The words “a”, “an”, and “the” as used herein mean “at least one” unless otherwise specifically indicated.

The (2S,3S)-2,3-butanediol dehydrogenase of the present invention can use NAD⁺ as coenzyme, preferentially oxidizes the hydroxyl groups of 2,3-butanediol in (S)-configuration and produces (2S,3S)-2,3-butanediol through the reduction of 2,3-butanedione using NADH as a coenzyme.

According to the present invention, the oxidative activity on (2S,3S)-2,3-butanediol and reductive activity on 2,3-butanedione of an enzyme can be confirmed as follows.

(1) Method for Measuring the Oxidative Activity on (2S,3S)-2,3-butanediol:

React a reaction solution containing 50 mM glycine-sodium hydroxide buffer (pH 11.0), 2.5 mM NAD⁺, 50 mM (2S,3S)-2,3-butanediol and the enzyme at 30° C. Then, measure the increase of absorbance at 340 nm relative to the increase of NADH. 1 U is defined as the amount of enzyme that catalyzes a 1-μmol increase of NADH in 1 minute.

(2) Method of Measuring the Reductive Activity on 2,3-butanedione:

React a reaction solution containing 50 mM potassium phosphate buffer (pH 5.5), 0.2 mM NADH, 20 mM 2,3-butanedione and the enzyme at 30° C. Then, measure the decrease of absorbance at 340 nm relative to the decrease of NADH. 1 U is defined as the amount of enzyme that catalyzes a 1-μmol decrease of NADH in 1 minute. The protein can be quantified by a dye binding method using BioRad protein assay kit.

The activation effect by a divalent ion can be measured as described above by adding 1 mM of a metal ion into the above-mentioned reaction solution. Herein, the phrase “the enzymatic activity was not substantially activated” refers to cases wherein the relative activity upon the addition of metal ion does not exceed 120, taking the activity without the addition of metal ion as 100.

In the present invention, the phrase “(2S,3S)-2,3-butanediol dehydrogenase ‘preferentially’ oxidizes the (2S,3S)-hydroxyl groups of 2,3-butanediol” means that when the enzyme activity of (2S,3S)-2,3-butanediol dehydrogenase on 2,3-butanediol in the (2S,3S)-configuration is taken as 100, the enzyme activity towards 2,3-butanediols in the (2R,3R)-configuration and meso configuration is 20 or less, preferably 10 or less, and more preferably 5 or less.

Furthermore, herein, the phrase “(2S,3S)-2,3-butanediol dehydrogenase ‘preferentially’ oxidizes the hydroxyl group of 2-butanol in the (S)-configuration” means that when the enzyme activity of (2S,3S)-2,3-butanediol dehydrogenase on2-butanol in the (S)-configuration is taken as 100, the enzyme activity on 2-butanol in the (R)-configuration is 20 or less, preferably 10 or less, and more preferably 5 or less.

Additionally, in the present invention, the phrase “(2S,3S)-2,3-butanediol dehydrogenase ‘preferentially’ oxidizes the hydroxyl groups of 1,2-butanediol in the (S) configuration” means that when the enzyme activity of (2S,3S)-2,3-butanediol dehydrogenase on 1,2-butanediol in the (S)-configuration is taken as 100, the enzyme activity on 1,2-butanediol in the (R)-configuration is 20 or less, preferably 10 or less, and more preferably 5 or less.

A (2S,3S)-2,3-butanediol dehydrogenase having the above-mentioned physicochemical properties can be purified, for example, from a culture of bacteria of the genus Zoogloea. Among the bacteria of the genus Zoogloea, Zoogloea ramigera in particular has excellent ability to produce the (2S,3S)-2,3-butanediol dehydrogenase of the present invention. Zoogloea ramigera that can be used to yield the (2S,3S)-2,3-butanediol dehydrogenase of this invention can be obtained, for example, as DSM 287 from Deutsche Sammlung von Mikroorganismen und Zellkulturen GmbH.

The above-mentioned microorganism can be cultured in a medium that is generally used for the cultivation of fungi, such as YPD medium (containing 1% yeast extract, 1% peptone and 2% glucose (pH 6.0)). To produce the (2S,3S)-2,3-butanediol dehydrogenase of the present invention, the methanol or glycerol in the YPD medium may be substituted with glucose.

The resulting bacterial cells are then disrupted in a buffer containing reducing agents, such as, for example, 2-mercaptoethanol, and protease inhibitors, such as, for example, phenylmethanesulfonyl fluoride (PMFS) and ethylenediaminetetraacetic acid (hereinafter abbreviated as EDTA), using physical impact of glass beads, or through the application of high pressure (e.g., using Minilab or French press) to obtain a cell-free extract. The enzyme of the present invention can be purified from the cell-free extract by properly combining solubility-dependent protein fractionation (precipitation by organic solvents such as, for example, acetone and dimethylsulfoxide, or salting out with ammonium sulfate); cation exchange chromatography; anion exchange chromatography; gel filtration; hydrophobic chromatography; and affinity chromatography using chelate, dye or antibody. For example, the cell-free extract can be purified to an almost single band in electrophoresis by the combined use of column-chromatographic procedures, such as, for example, blue-Sepharose, phenyl-Sepharose, Mono-Q (all provided by Amersham Biosciences) and hydroxyapatite (KOKEN).

The Zoogloea ramigera-derived (2S,3S)-2,3-butanediol dehydrogenase of the present invention is an enzyme protein having at least the following physicochemical properties of (1) to (3), and preferably those of (1) to (5):

(1) Function:

produces (S)-acetoin by acting on (2S,3S)-2,3-butanediol using NAD⁺ as the coenzyme; and produces (2S,3S)-2,3-butanediol by reducing 2,3-butanedione using NADH as the coenzyme;

(2) Substrate Specificity:

uses NAD⁺ as the coenzyme for oxidation reaction, and NADH as the coenzyme for reduction reaction. Furthermore, preferentially oxidizes (2S,3S)-2,3-butanediol among the three isomers of 2,3-butanediol;

(3) Activation by Divalent Ions:

substantially not activated by Mg²⁺, Ca²⁺, Ba²⁺, Co²⁺ or Mn²⁺ ion;

(4) Substrate Specificity:

preferentially oxidizes the hydroxyl group of 2-butanol in the (S)-configuration; and

(5) Substrate Specificity:

preferentially oxidizes the hydroxyl groups of 1,2 -propanediol in the (S)-configuration.

Furthermore, the enzyme of this invention can be characterized by the following properties of (6) to (8):

(6) Optimal pH:

the optimal pH of (2S,3S)-2,3-butanediol oxidation reaction is about 11.0;

(7) Molecular Weight:

the molecular weight of the subunit, according to sodium dodecyl sulfate-polyacrylamide gel electrophoresis (hereinafter abbreviated as SDS-PAGE), is approximately 27,000; and

(8) Optimal Temperature:

the optimal temperature is about 45° C.

The (2S,3S)-2,3-butanediol dehydrogenase derived from Zoogloea ramigera substantially does not utilize NADP⁺ as a coenzyme in oxidation reaction and NADPH as a coenzyme in reduction reaction. However, regardless of the usability of NADP⁺ and NADPH, enzymes having the above-mentioned physicochemical properties (1) to (3), preferably (1) to (5), and even more preferably (1) to (8) are included in the present invention.

The present invention further relates to polynucleotides encoding a (2S,3S)-2,3-butanediol dehydrogenase and homologues thereof. Herein, the polynucleotides may be artificial molecules containing artificial nucleotide derivatives or naturally occurring polynucleotides such as DNAs and RNAs. Furthermore, the polynucleotides of the present invention can be chimeric molecules between DNAs and RNAs. A polynucleotide encoding the (2S,3S)-2,3-butanediol dehydrogenase of the present invention preferably comprises, for example, the nucleotide sequence of SEQ ID NO: 1. The nucleotide sequence of SEQ ID NO: 1 encodes a protein comprising the amino acid sequence of SEQ ID NO: 2. A protein comprising this amino acid sequence constitutes a preferred embodiment of the (2S,3S)-2,3-butanediol dehydrogenase according to the present invention.

A homologue of a polynucleotide encoding the (2S,3S)-2,3-butanediol dehydrogenase of the present invention includes a polynucleotide encoding a protein comprising the amino acid sequence of SEQ ID NO: 2, in which one or more amino acids are deleted, substituted, inserted and/or added, yet which retains the above physicochemical properties (1) to (3). Those skilled in the art can readily obtain such homologue polynucleotides by properly introducing substitution, deletion, insertion and/or addition mutations into the polynucleotide of SEQ ID NO: 1 by site-directed mutagenesis (Nucleic Acids Res. 10, 6487 (1982); Methods in Enzymol. 100, 448 (1983); Molecular Cloning 2nd Ed., Cold Spring Harbor Laboratory Press (1989); PCR A Practical Approach, pp. 200, IRL Press (1991)) or the like.

Within the amino acid sequence of SEQ ID NO: 2, the allowed number of mutated amino acid residues is, for example, about 100 or less, normally about 50 or less, preferably about 30 or less, more preferably about 15 or less, even more preferably about 10 or less, or about 5 or less. Alternatively, the mutated amino acid residues preferably constitute no more than 5% of the total coding sequence. Mutations of amino acid residues in the homolog of the present invention are preferably conservative substitutions. Generally, to maintain the function of a protein, amino acids having similar characteristics to the amino acid to be substituted are preferably used for substitution. This type of amino acid substitution is called conservative substitution.

For example, Ala, Val, Leu, Ile, Pro, Met, Phe and Trp are all categorized into nonpolar amino acids and, therefore, have similar properties to each other. Uncharged amino acids include Gly, Ser, Thr, Cys, Tyr, Asn and Gln. Acidic amino acids include Asp and Glu. Furthermore, basic amino acids include Lys, Arg and His.

In addition, a homologue of a polynucleotide of the present invention encompasses a polynucleotide hybridizing under stringent conditions to a polynucleotide comprising the nucleotide sequence of SEQ ID NO: 1, and which encodes a protein having the above physicochemical properties (1) to (3). The phrase “a polynucleotide hybridizing under stringent conditions” refers to a polynucleotide that hybridizes when one or more DNAs having sequences at least about 20, preferably at least about 30, for example, about 40, about 60 or about 100 constitutive arbitrarily selected nucleotides of SEQ ID NO: 1, are used as a probe DNA, and for example, using ECL direct nucleic acid labeling and detection system (Amersham Biosciences) under conditions described in the manual (e.g., washing at 42° C. in primary wash buffer containing 0.5×SSC).

Hybridization can be carried out according to conventional methods using a nitrocellulose membrane, nylon membrane or such (Sambrook et al. (1989) Molecular Cloning, Cold Spring Harbor Laboratories; Ausubel, F. M. et al. (1994) Current Protocols in Molecular Biology, Greene Publishing Associates/John Wiley and Sons, New York. N.Y.).

A more specific example of the above-mentioned stringent condition comprises a condition wherein the hybridization is performed overnight in a solution comprising 6×SSC, 0.5% (W/V) SDS, 100 μg/mL denatured salmon sperm DNA, 5× Denhardt's solution (1× Denhardt's solution comprises 0.2% polyvinylpyrrolidone, 0.2% bovine serum albumin and 0.2% ficoll) at about 45° C., preferably at about 55° C., more preferably at about 60° C. and even more preferably at about 65° C., and carrying out the wash after hybridization at the same temperature used for the hybridization, three times in 4×SSC and 0.5% SDS for 20 minutes. An example of a more preferred stringent condition comprises the step of performing the wash after hybridization at the same temperature used for the hybridization, twice in 4×SSC and 0.5% SDS for 20 minutes, and once in 2×SSC and 0.5% SDS for 20 minutes. An even more preferred stringent condition comprises the step of performing the wash after hybridization at the same temperature used for the hybridization, twice in 4×SSC and 0.5% SDS for 20 minutes, and then once in 1×SSC and 0.5% SDS for 20 minutes. A still more preferred stringent condition comprises the step of performing the wash after hybridization at the same temperature used for the hybridization, once in 2×SSC and 0.5% SDS for 20 minutes, once in 1×SSC and 0.5% SDS for 20 minutes, and then once in 0.5×SSC and 0.5% SDS for 20 minutes. A yet even more preferred stringent condition comprises the step of performing the wash after hybridization at the same temperature used for the hybridization, once in 2×SSC and 0.5% SDS for 20 minutes, once in 1×SSC and 0.5% SDS for 20 minutes, once in 0.5×SSC and 0.5% SDS for 20 minutes, and then once in 0.1×SSC and 0.5% SDS for 20 minutes.

Furthermore, the polynucleotide homologues of the present invention include a polynucleotide encoding a protein having at least about 70%, preferably at least about 80% or about 90%, and more preferably about 95% or higher homology to the amino acid sequence of SEQ ID NO: 2. Homology search of proteins can be achieved, for example, on the Internet using a program such as BLAST and FASTA, for example, in databases related to amino acid sequences of proteins, such as DAD, SWISS-PROT and PIR; databases related to DNA sequences, such as DDBJ, EMBL and GenBank; and databases related to deduced amino acid sequences based on DNA sequences.

A homology search using the BLAST program was performed on the DAD database using the amino acid sequence of SEQ ID NO: 2. As a result, it was discovered that acetoin reductase of Klebsiella pneumoniae showed the highest homology among the known proteins. This acetoin reductase is known to specifically act on meso-butanediol alone, and doesnot act on (2S,3S)-2,3-butanediol (J. Ferment. Technol. 83, 32-37 (1997)). The homology of the amino acid sequence of SEQ ID NO: 2 to this acetoin reductase was 56% Identity and 67% Positive. 70% or more homology in the present invention refers to, for example, the homology value for Identity using the BLAST program.

The present invention relates to proteins comprising the amino acid sequence of SEQ ID NO: 2. The present invention further includes homologues of the protein comprising the amino acid sequence of SEQ ID NO: 2.

A homologue of the (2S,3S)-2,3-butanediol dehydrogenase of the present invention refers to a protein comprising the amino acid sequence of SEQ ID NO: 2 in which one or more amino acids are deleted, substituted, inserted and/or added, yet which is functionally equivalent to a protein comprising the amino acid sequence of SEQ ID NO: 2. In the context of the present invention, the phrase “functionally equivalent to a protein comprising the amino acid sequence of SEQ ID NO: 2” means that the protein has the above-mentioned physicochemical properties (1) to (3). Those skilled in the art can obtain a polynucleotide encoding such a homologue of the (2S,3S)-2,3-butanediol dehydrogenase by properly introducing substitution, deletion, insertion and/or addition mutations into the DNA of SEQ ID NO: 1 by site-directed mutagenesis (Nucleic Acids Res. 10, 6487 (1982); Methods in Enzymol. 100, 448 (1983); Molecular Cloning 2nd Ed., Cold Spring Harbor Laboratory Press (1989); PCR A Practical Approach, 200, IRL Press(1991)) or such. It is possible to obtain a homologue of the (2S,3S)-2,3-butanediol dehydrogenase of SEQ ID NO: 2, by introducing and expressing the polynucleotide encoding the homologue of (2S,3S)-2,3-butanediol dehydrogenase into a host.

Furthermore, the homologue of the (2S,3S)-2,3-butanediol dehydrogenase of the present invention includes a protein having at least about 70%, preferably at least about 80% or about 90%, and more preferably about 95% or higher homology to the amino acid sequence of SEQ ID NO: 2. Homology search of a protein can be achieved, for example, on the Internet using a program such as BLAST and FASTA, for example, in databases related to amino acid sequences of proteins, such as DAD, SWISS-PROT and PIR; databases related to DNA sequences, such as DDBJ, EMBL and GenBank; and databases related to deduced amino acid sequences based on DNA sequences.

Polynucleotides encoding the (2S,3S)-2,3-butanediol dehydrogenase of the present invention can be isolated, for example, by following methods.

PCR primers are designed based on the nucleotide sequence of SEQ ID NO: 1, and a polynucleotide of the present invention can be obtained by conducting PCR using genomic DNA or cDNA library of an enzyme-producing strain as the template.

Moreover, a polynucleotide of the present invention can be obtained through colony hybridization, plaque hybridization and so on. Such hybridization can be performed using the obtained DNA fragment as a probe, and cDNA library or a library obtained by transforming E. coli with phage, plasmid, etc. that are introduced with restriction enzyme digestion product of the genomic DNA of an enzyme-producing strain.

It is also possible to obtain a polynucleotide of the present invention by, first, analyzing the nucleotide sequence of the obtained DNA fragment by PCR and designing PCR primers to elongate the fragment to the outside of the known sequence. After digesting the genomic DNA of an enzyme-producing strain with an appropriate restriction enzyme, reverse PCR that utilizes the self-cyclization reaction of DNA using the DNA as the template (Genetics 120, 621-623 (1988)), the RACE method (Rapid Amplification of cDNA End, “PCR experimental manual”, 25-33, HBJ press) and such is performed.

The polynucleotides of the present invention include not only genomic DNAs or cDNAs cloned by the above-mentioned methods but also synthesized polynucleotides.

An isolated polynucleotide encoding the (2S,3S)-2,3-butanediol dehydrogenase of the present invention may be inserted into a known expression vector to provide a (2S,3S)-2,3-butanediol dehydrogenase-expressing vector.

Furthermore, by culturing cells transformed with such an expression vector, the (2S,3S)-2,3-butanediol dehydrogenase of the present invention can be obtained from the transformed cells.

Herein, there is no restriction on the microorganism to be transformed for expressing (2S,3S)-2,3-butanediol dehydrogenase whose electron acceptor is NAD⁺, so long as the microorganism can be transformed with a recombinant vector comprising a polynucleotide encoding a polypeptide having (2S,3S)-2,3-butanediol dehydrogenase activity whose electron acceptor is NAD⁺ and that can express active (2S,3S)-2,3-butanediol dehydrogenase. Suitable microorganisms are those for which a host-vector system is available and include, but are not limited to:

bacteria such as:

the genus Escherichia,
the genus Bacillus,
the genus Pseudomonas,
the genus Serratia,
the genus Brevibacterium,
the genus Corynebacterium,
the genus Streptococcus, and
the genus Lactobacillus;
   actinomycetes such as:
the genus Rhodococcus, and
the genus Streptomyces;
   yeasts such as:
the genus Saccharomyces,
the genus Kluyveromyces,
the genus Schizosaccharomyces,
the genus Zygosaccharomyces,
the genus Yarrowia,
the genus Trichosporon,
the genus Rhodosporidium,
the genus Pichia, and
the genus Candida; and
   fungi such as:
the genus Neurospora,
the genus Aspergillus,
the genus Cephalosporium, and
the genus Trichoderma.

Preparation of a transformant and construction of a recombinant vector suitable as a host can be carried out using techniques that are commonly used in the fields of molecular biology, bioengineering and genetic engineering (for example, see Sambrook et al., “Molecular Cloning”, Cold Spring Harbor Laboratories). In order to express in a microorganism a gene encoding the (2S,3S)-2,3-butanediol dehydrogenase of the present invention whose electron donor is NAD⁺, it is necessary to introduce the DNA into a plasmid vector or phage vector that is stable in the microorganism and let the genetic information transcribe and translate.

Therefore, a promoter, a unit for regulating transcription and translation, is preferably incorporated upstream of the 5′ end of the DNA of the present invention; similarly, a terminator is preferably incorporated downstream of the 3′ end of the DNA. The promoter and the terminator should be functional in the microorganism to be utilized as a host. Available vectors, promoters and terminators for the above-mentioned various microorganisms are described in detail in “Fundamental Course in Microbiology (8): Genetic Engineering”, Kyoritsu Shuppan, and specifically for yeasts in Adv. Biochem. Eng. 43, 75-102 (1990) and Yeast 8, 423-488 (1992).

For example, for the genus Escherichia, in particular, for Escherichia coli, suitable plasmids include, but are not limited to, the pBR series and pUC series plasmids. Suitable promoters include, but are not limited to, those derived from lac (that of the β-galactosidase gene), trp (that of the tryptophan operon), tac and trc (chimeras of lac and trp), PL and PR of k phage, etc. Suitable terminators include, but are not limited to, those derived from trpA, phages, rrnB ribosomal RNA, etc. Among these, the pSE420D vector (described in Unexamined Published Japanese Patent Application No. (JP-A) 2000-189170), which is constructed by partially modifying the multicloning site of commercially available pSE420 (Invitrogen), can be preferably used.

For the genus Bacillus, suitable vectors include, but are not limited to, the pUB110 series and pC194 series plasmids. The vectors can be integrated into host chromosome. Suitable promoters and terminators include, but are not limited to, apr (that of alkaline protease), npr (that of neutral protease), amy (that of α-amylase), etc.

For the genus Pseudomonas, host-vector systems for Pseudomonas putida, Pseudomonas cepacia and such have been developed. A broad-host-range vector, pKT240, (comprising RSF1010-derived genes required for autonomous replication) based on TOL plasmid, which is involved in decomposition of toluene compounds, is particularly suitable. The promoter and terminator derived from a lipase gene (JP-A Hei 5-284973) are also quite suitable.

For the genus Brevibacterium, in particular, for Brevibacterium lactofermentum, suitable plasmid vectors include, but are not limited to, pAJ43 (Gene 39, 281 (1985)). Promoters and terminators used for Escherichia coli can be utilized without any modification for Brevibacterium.

For the genus Corynebacterium, in particular, for Corynebacterium glutamicum, plasmid vectors such as pCS11 (JP-A Sho 57-183799) and pCB101 (Mol. Gen. Genet. 196, 175(1984)) are suitable.

For the genus Streptococcus, plasmid vectors such as pHV1301 (FEMS Microbiol. Lett. 26, 239 (1985)) and pGK1 (Appl. Environ. Microbiol. 50, 94 (1985)) can be used.

For the genus Lactobacillus, plasmid vectors such as pAMP1 (J. Bacteriol. 137, 614 (1979)), which was developed for the genus Streptococcus, can be utilized; and promoters that are used for Escherichia coli are also suitable.

For the genus Rhodococcus, plasmid vectors isolated from Rhodococcus rhodochrous are suitable (J. Gen. Microbiol. 138, 1003 (1992)).

For the genus Streptomyces, plasmids can be constructed in accordance with the method as described in “Genetic Manipulation of Streptomyces: A Laboratory Manual” (Cold Spring Harbor Laboratories (1985)) by Hopwood et al. In particular, for Streptomyces lividans, pIJ486 (Mol. Gen. Genet. 203, 468-478, 1986) , pKC1064 (Gene 103, 97-99 (1991)) and pUWL-KS (Gene 165, 149-150 (1995)) are suitable. The same plasmids can also be utilized for Streptomyces virginiae (Actinomycetol. 11, 46-53 (1997)).

For the genus Saccharomyces, in particular, for Saccharomyces cerevisiae, the YRp series, YEp series, YCp series and YIp series plasmids are particularly suitable. Integration vectors (see EP 537456, and so on), which are integrated into the chromosome via homologous recombination with multicopy-ribosomal genes, allow the introduction of a gene of interest in multicopy and the incorporated gene is stably maintained in the microorganism. Thus, these types of vectors are highly useful. Suitable promoters and terminators may be derived, for example, from genes encoding alcohol dehydrogenase (ADH), glyceraldehyde-3-phosphate dehydrogenase (GAPDH), acid phosphatase (PHO), β-galactosidase (GAL), phosphoglycerate kinase (PGK), enolase (ENO), etc.

For the genus Kluyveromyces, in particular, for Kluyveromyces lactis, suitable plasmids include, but are not limited to, those such as the 2-μm series plasmids derived from Saccharomyces cerevisiae, pKD1 series plasmids (J. Bacteriol. 145, 382-390(1981)), plasmids derived from pGKl1 that is involved in the killer activity, Kluyveromyces autonomous replication sequence (KARS) series plasmids, and plasmids (see EP 537456, and so on) that can be integrated into the chromosome via homologous recombination with a ribosomal DNA. Promoters and terminators derived from ADH, PGK and such are also suitable.

For the genus Schizosaccharomyces, one may use plasmid vectors comprising autonomous replication sequence (ARS) derived from Schizosaccharomyces pombe and auxotrophy-complementing selectable markers derived from Saccharomyces cerevisiae (Mol. Cell. Biol. 6, 80 (1986)). Promoters such as the ADH promoter derived from Schizosaccharomyces pombe are also suitable (EMBO J. 6, 729 (1987)). In particular, pAUR224 is commercially available from TaKaRa Shuzo Co., Ltd. and can be readily used for the genus Schizosaccharomyces.

For the genus Zygosaccharomyces, plasmids originating from pSB3 (Nucleic Acids Res. 13, 4267 (1985)) derived from Zygosaccharomyces rouxii, for example, are suitable. One may also use promoters such as the PHOS promoter derived from Saccharomyces cerevisiae and the GAP-Zr (Glyceraldehyde-3-phosphate dehydrogenase) promoter derived from Zygosaccharomyces rouxii (Agric. Biol. Chem. 54, 2521 (1990)).

A host-vector system has been developed for Pichia angusta (previously called Hansenula polymorpha) among the genus Pichia. Suitable vectors include, but are not limited to, Pichia angusta-derived genes involved in autonomous replication (HARS1 and HARS2); however, such vectors are relatively unstable. Therefore, multi-copy integration of a gene into the chromosome is effective (Yeast 7, 431-443 (1991)). Promoters of alcohol oxidase (AOX) and formic acid dehydrogenase (FDH), which are induced by methanol and such, are also suitable. Another host-vector system wherein Pichia-derived genes involved in autonomous replication (PARS1 and PARS2) are used in Pichia pastoris and such has been developed (Mol. Cell. Biol. 5, 3376 (1985)). In this system, strong promoters such as AOX that is inducible by high-density cultivation and methanol are suitable (Nucleic Acids Res. 15, 3859 (1987)).

With regard to the genus Candida, host-vector systems have been developed for Candida maltosa, Candida albicans, Candida tropicalis, Candida utilis, etc. An ARS originating from Candida maltosa has been cloned (Agric. Biol. Chem. 51, 1587 (1987)) and a vector using the sequence has been developed for Candida maltosa. Furthermore, a highly efficient promoter unit has been developed for chromosome-integration vectors of Candida utilis (JP-A Hei. 08-173170).

For the genus Aspergillus, Aspergillus niger, Aspergillus oryzae and such have intensively been studied among fungi. Thus, plasmid vectors and chromosome-integration vectors are suitable, as well as promoters derived from an extracellular protease gene and amylase gene (Trends in Biotechnology 7, 283-287 (1989)).

For the genus Trichoderma, host-vector systems have been developed for Trichoderma reesei, and promoters such as that derived from an extracellular cellulase gene are suitable (Biotechnology 7, 596-603(1989)).

Apart from microorganisms, there are various host-vector systems developed for plants and animals. In particular, the systems including those for large-scale production of foreign proteins in insects, such as silkworm (Nature 315, 592-594 (1985)) , and plants such as rapeseed, maize, potato, etc. are preferably employed.

Microorganisms capable of producing (2S,3S)-2,3-butanediol dehydrogenase to be utilized in the present invention include all strains belonging to the genus Zoogloea that are capable of producing NAD⁺-dependent (2S,3S)-2,3-butanediol dehydrogenase, mutants and variants thereof, as well as transformants that have acquired the ability to produce the enzyme of the present invention through genetic manipulation.

The present invention relates to the use of the above-mentioned (2S,3S)-2,3-butanediol dehydrogenase in the production of alcohol, particularly (2S,3S)-2,3-butanediol, via the reduction of ketone. The enzyme reaction of interest can be carried out by contacting the enzyme molecule, treated products thereof, cultures containing the enzyme molecule or living transformants, such as microorganisms producing the enzyme, with a reaction solution. The form of contacting the enzyme and the reaction solution are not limited to these specifically disclosed examples.

The reaction solution preferably comprises a substrate and NADH, a coenzyme required for the enzyme reaction, both dissolved in a suitable solvent which yields an environment desirable for enzyme activity. Treated products of a microorganism containing the (2S,3S)-2,3-butanediol dehydrogenase in accordance with the present invention specifically include microorganisms wherein the permeability of the cell membrane has been altered by a detergent or an organic solvent, such as, for example, toluene; and cell-free extracts obtained by lysing cells, for example, using glass beads or an enzyme treatment, or partially purified material thereof.

An example of a ketone used in the method for producing alcohol of the present invention is represented by formula I or II shown below. By reacting the (2S,3S)-2,3-butanediol dehydrogenase of the present invention with such a ketone, the keto group is sterospecifically reduced and an optically active alcohol is produced.

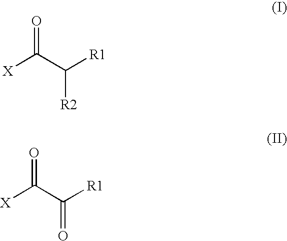

In formula I or II, X denotes a hydrogen atom, halogen atom or hydroxyl group; R1 denotes a hydrogen atom, a substituted or unsubstituted C1-C6 straight chain or branched chain alkyl group, a substituted or unsubstituted C1-C6 straight chain or branched chain alkenyl group, or a substituted or unsubstituted C1-C6 straight chain or branched chain alkynyl group; and R2 denotes a hydrogen atom, carbonyl group, hydroxyl group, a substituted or unsubstituted C1-C6 straight chain or branched chain alkyl group, a substituted or unsubstituted C1-C6 straight chain or branched chain alkenyl group, or a substituted or unsubstituted C1-C6 straight chain or branched chain alkynyl group. R1 and R2 may be bound to form a ring.

More specifically, exemplary ketones that find utility in the method for producing alcohol of the present invention include, but are not limited to, 2,3-butanedione, 2,3-pentanedione, acetoin and 2-butanone. Using such a compound as a substrate, (2S,3S)-2,3-butanediol, (2S,3S)-2,3-pentanediol, (S)-1,2-propanediol and (S)-2-butanol, can be respectively synthesized.

The present invention relates to producing ketones via alcohol oxidation reaction by the above-mentioned (2S,3S)-2,3-butanediol dehydrogenase. The enzyme reaction of interest can be carried out by contacting the enzyme molecule, treated products thereof, cultures comprising the enzyme molecule, or living transformants, such as microorganisms producing the enzyme, with a reaction solution. The form of contacting the enzyme and reaction solution are not limited to these specifically disclosed examples.

The reaction solution preferably comprises a substrate and an NAD⁺, a coenzyme required for the enzyme reaction, both dissolved in a suitable solvent which yields an environment desirable for the enzyme activity. The treated products of microorganisms containing the (2S,3S)-2,3-butanediol dehydrogenase in accordance with the present invention specifically include microorganisms wherein the permeability of the cell membrane has been altered by a detergent or an organic solvent, such as, for example, toluene; and cell-free extracts obtained by lysing cells using, for example, glass beads or an enzyme treatment, or partially purified material thereof.

Alcohols to be used in the method for producing ketones in accordance with the present invention include (2S,3S)-2,3-butanediol; (S)-acetoin can be synthesized from this compound.

Using a racemic alcohol as a substrate, the above-mentioned (2S,3S)-2,3-butanediol dehydrogenase can also be used for the production of optically active alcohols in the present invention, where the production utilizes the asymmetric oxidizing ability of the enzyme. Specifically, an optically active alcohol is produced through the preferential oxidation of one of the optical isomers with the enzyme of this invention, thereby yielding the optically active alcohol. More specifically, the (2S,3S)-2,3-butanediol dehydrogenase of the present invention together with NAD⁺ is reacted with racemic 2-butanol or racemic 1,2-propanediol in which the (S)-form and (R)-form are mixed.

The (2S,3S)-2,3-butanediol dehydrogenase of the present invention has excellent stereoselectivity specifically such that it acts on an (S)-form alcohol to oxidize it into a ketone, but fails to act on an (R)-form alcohol. Therefore, the proportion of the (R)-form eventually increases in the reaction. By separating the (R)-form which accumulates in this manner, eventually, the (R)-form alcohol can be collected from a racemate. This way, (R)-2-butanol or (R)-1,2-propanediol may be yielded from racemic 2-butanol or racemic 1,2-propanediol, respectively.

The phrase “optically active alcohol” in the context of the present invention refers to an alcohol containing more of a certain optical isomer as compared to the other optical isomer, or an alcohol containing only a particular optical isomer. Furthermore, in the context of the present invention, the phrase “optical isomer” is also generally called an “optically active substance” or an “enantiomer”.

The regeneration of NADH from NAD⁺ that is generated from NADH in association with the above reduction reaction can be achieved using a microorganism having the ability to reduce NAD⁺ (glycolytic pathway, assimilation pathway for C1 compounds of methylotroph, and so on). The NAD⁺ reducing ability of a microorganism can be enhanced by adding glucose, ethanol, formic acid or such into the reaction system. Alternatively, the regeneration of NADH can also be achieved by adding microorganisms capable of generating NADH from NAD⁺, treated products thereof, or enzymes into the reaction system. For example, the regeneration of NADH can be accomplished using microorganisms containing glucose dehydrogenase, formic acid dehydrogenase, alcohol dehydrogenase, amino acid dehydrogenase, organic acid dehydrogenase (e.g., malate dehydrogenase) and such; treated products thereof; or partially or fully purified enzymes. These components for the reaction required for NADH regeneration can be added to, added after immobilization to, or contacted via an NADH-permeable membrane with the reaction system for producing alcohols in accordance with the present invention.

In some cases, when living cells of microorganisms transformed with recombinant vectors comprising the polynucleotide of the present invention are utilized in the above-mentioned method for producing alcohols, additional reaction systems for the regeneration of NADH may be unnecessary. Specifically, when a microorganism having high activity of regenerating NADH is used, an efficient reaction can be achieved in the reduction reaction using transformants without the addition of the enzyme for the regeneration of NADH. Furthermore, it is possible to more efficiently express the NADH regenerating enzyme and NAD⁺-dependent (2S,3S)-2,3-butanediol dehydrogenase, and thus achieve an efficient reduction reaction, by co-introducing a gene of glucose dehydrogenase, formic acid dehydrogenase, alcohol dehydrogenase, amino acid dehydrogenase, organic acid dehydrogenase (e.g., malate dehydrogenase) or such, which are usable to regenerate NADH, together with a polynucleotide encoding the NADH-dependent (2S,3S)-2,3-butanediol dehydrogenase of the present invention into a host.

To introduce two or more genes into a host, the following methods may be utilized: a method wherein a host is transformed with multiple recombinant vectors constructed by separately inserting the genes into multiple vectors comprising different replication origins to avoid incompatibility; a method wherein both genes are inserted into a single vector; and a method wherein either or both of the genes are integrated into the chromosome.

When multiple genes are introduced into a single vector, it is possible to ligate regions involved in regulating the expression, such as promoter and terminator, to each gene, or to express the genes in a form of operon that contains multiple cistrons, such as the lactose operon.

The reduction reaction using the enzyme of the present invention can be performed in water; organic solvent that is immiscible with water, for example, organic solvents such as ethyl acetate, butyl acetate, toluene, chloroform and n-hexane; or a heterogeneous two-solvent system of organic solvent and aqueous solvent.

The reaction in accordance with the present invention can be conducted at about 4° C. to 60° C., preferably about 15° C. to 30° C., at pH of about 3 to 11, preferably pH of about 6 to 9.5, at a substrate concentration of about 0.01% to 90%, preferably about 0.1% to 30%. As needed, coenzyme NAD⁺ or NADH may be added at a concentration of about 0.001 mM to 100 mM, preferably about 0.01 mM to 10 mM in the reaction system. Furthermore, the substrate can be added at the start of the reaction; however, it is preferable to continuously or stepwise add the substrate so that its concentration does not become too high in the reaction mixture.

In the regeneration of NADH, for example, glucose is added to the reaction system when glucose dehydrogenase is used; formic acid is added when formic acid dehydrogenase is used; and ethanol or isopropanol is added when alcohol dehydrogenase is used. These compounds can be added in about 0.1 to 20 fold excess, preferably about 1 to 5 fold excess over the substrate ketone at the molar ratio. On the other hand, it is possible to add the enzymes for regenerating NADH, such as glucose dehydrogenase, formic acid dehydrogenase and alcohol dehydrogenase, in about 0.1 to 100 fold excess, preferably about 0.5 to 20 fold excess in enzyme activity as compared with the NADH-dependent carbonyl dehydrogenase of the present invention.

The purification of alcohol generated by the reduction of ketones according to the present invention can be performed by properly combining centrifugation, separation through membrane treatment or such, extraction by solvent, distillation, and so on of fungal cells and proteins.

For example, in the interest of (2S, 3S)-2,3-butanediol, highly purified (2S,3S)-2,3-butanediol dehydrogenase can be prepared by separating a reaction mixture containing cells of a microorganism by centrifugation to remove the cells, removing proteins by ultrafiltration, adding solvent, such as ethyl acetate, to the filtrate for the extraction of (2S,3S)-2,3-butanediol into the solvent phase followed by phase separation, and then distillation.

According to the present invention, an NAD⁺-dependent (2S,3S)-2,3-butanediol dehydrogenase useful for the production of optically active alcohol or such, and polynucleotides encoding the enzyme are provided. Methods for efficiently producing (2S,3S)-2,3-butanediol and (S)-2-butanol with high optical purity were provided by utilizing the enzyme. Since the (2S,3S)-2,3-butanediol dehydrogenase of the present invention is dependent on NAD⁺, which is more stable than NADP⁺, it can be conveniently used in industrial production processes.

The methods for producing (2S,3S)-2,3-butanediol and (S)-2-butanol of high optical purity according to the present invention are useful as methods for producing raw materials for liquid crystals, pharmaceutical agents, etc.

The following examples are presented to illustrate the present invention and to assist one of ordinary skill in making and using the same. The examples are not intended in any way to otherwise limit the scope of the invention.

Any patents, published patent applications and publications cited herein are incorporated by reference.

### EXAMPLES

Hereinafter, the present invention will be specifically described using the following Examples; however, the invention should not be construed as being limited thereto.

### Example 1

### Purification of (2S,3S)-2,3-butanediol dehydrogenase

Zoogloea ramigera strain DSM287 was cultured at 28° C. for 54 hours in 1.2 L of YPD medium that uses glycerol as a carbon source, and wet bacterial cells were prepared by centrifugation. Approximately 30 g of the obtained wet bacterial cells were suspended in a 50 mL solution of 50 mM potassium phosphate buffer (pH8.0) and 1 mM 2-mercaptoethanol. The cells were then disrupted using Multi-beads shocker (Yasui Kikai), and bacterial cell debris was removed by centrifugation to obtain cell-free extract. Protamine sulfate was added to this cell-free extract and nucleic acids were removed by centrifugation to yield the supernatant. Ammonium sulfate was added to this supernatant up to 30% saturation, and this was applied onto phenyl-sepharose column (2.6 cm×10 cm) equilibrated with standard buffer (10 mM Tris-HCl buffer (pH8.0) and 0.01% 2-mercaptoethanol) comprising 30% ammonium sulfate. After washing the column with the same buffer, gradient elution was performed with 30% to 0% saturated ammonium sulfate. Eluted fractions having (2S,3S)-2,3-butanediol dehydrogenase activity were collected and concentrated by ultrafiltration.

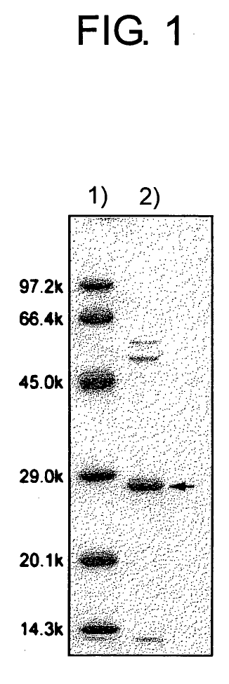

The concentrated enzyme solution was dialyzed against the standard buffer and then applied onto a MonoQ Column (1.6 cm×10 cm) equilibrated with this buffer. After washing with the same buffer, gradient elution was performed with 0 to 1 M sodium chloride, and active fractions were concentrated by ultrafiltration. The concentrated enzyme solution was dialyzed against the standard buffer, and then applied onto Blue Sepharose Column (1.6 cm×2.5 cm) equilibrated with this buffer. After washing with the same buffer, gradient elution was performed with 0 to 1 M sodium chloride and active fractions were concentrated by ultrafiltration. The concentrated enzyme solution was dialyzed against a 1 mM potassium phosphate buffer containing 0.01% 2-mercaptoethanol, and then applied onto a hydroxyapatite column (0.5 cm×10 cm) equilibrated with this buffer. After washing with the same buffer, gradient elution was performed with 1 mM to 350 mM potassium phosphate buffer. Analysis of active fractions by SDS-PAGE showed an almost single band (FIG. 1).

The specific activity for (2S,3S)-2,3-butanediol dehydrogenase of the purified enzyme was approximately 60.9 U/mg-protein. The purification procedure is summarized in Table 1.

TABLE 1

| | | Enzyme activity | Specific |
|---|---|---|---|
| Step | Protein (mg) | (U) | activity (U/mg) |
| Cell-free extract | 1,390 | 255 | 0.183 |
| Phenyl-Sepharose | 322.8 | 210 | 0.650 |
| Mono-Q | 6.71 | 54.8 | 8.16 |
| Blue Sepharose | 2.06 | 5.29 | 2.57 |
| Hydroxyapatite | 0.048 | 2.92 | 60.9 |

### Example 2

### Molecular Weight Determination of (2S,3S)-2,3-butanediol dehydrogenase

The molecular weight of the subunit of the enzyme obtained in Example 1 was determined to be 27,000 by SDS-PAGE.

### Example 3

### Optimal pH for Oxidation Reaction by (2S,3S)-2,3-butanediol dehydrogenase

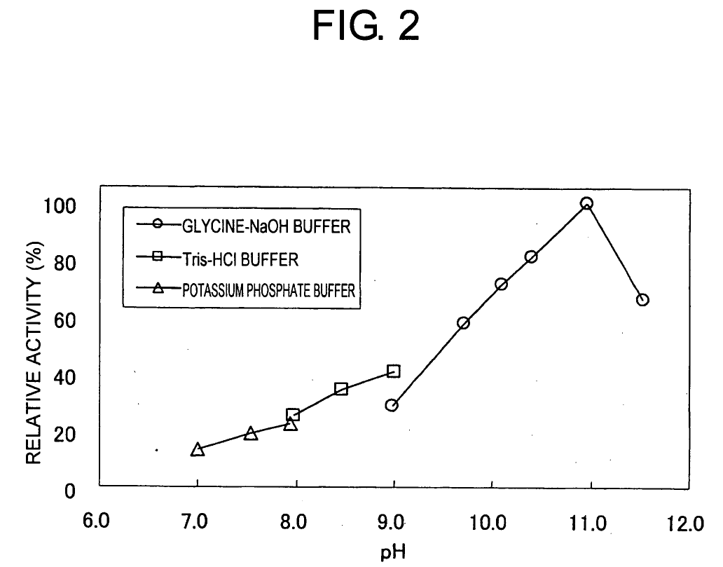

The activity of the enzyme, (2S,3S)-2,3-butanediol dehydrogenase, obtained in Example 1 was investigated by changing the pH using a potassium phosphate buffer, a Tris-HCl buffer and a glycine-sodium hydroxide buffer. The results are shown in FIG. 2. The enzyme activity is expressed as relative activity, taking the maximum activity as 100. The optimal pH for the oxidation reaction was 11.0.

### Example 4

### Optimal pH for Reduction Reaction by (2S,3S)-2,3-butanediol dehydrogenase

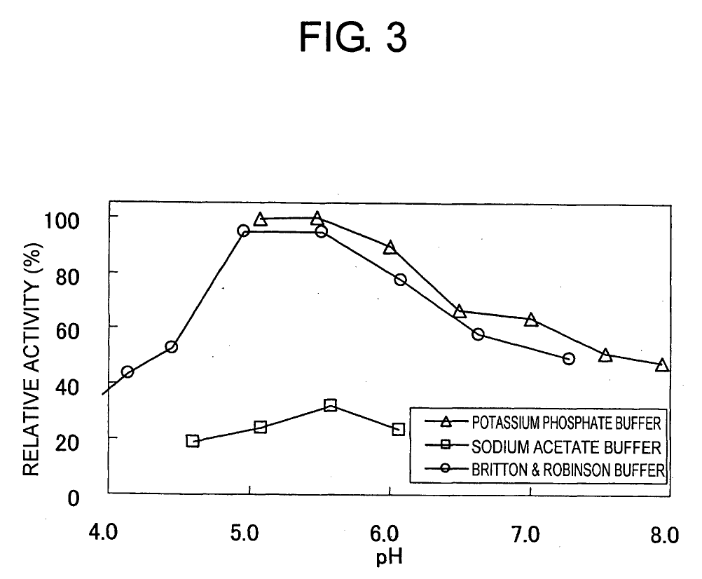

The activity of the enzyme, 2,3-butadione reductase, obtained in Example 1 was investigated by changing the pH using Britton and Robinson's universal buffer, a sodium acetate buffer and a potassium phosphate buffer. The results are shown in FIG. 3. The enzyme activity is expressed as relative activity, taking the maximum activity as 100. The optimal pH was 5.0 to 5.5 for the reduction reaction.

### Example 5

### Optimal Temperature for (2S,3S)-2,3-butanediol dehydrogenase function

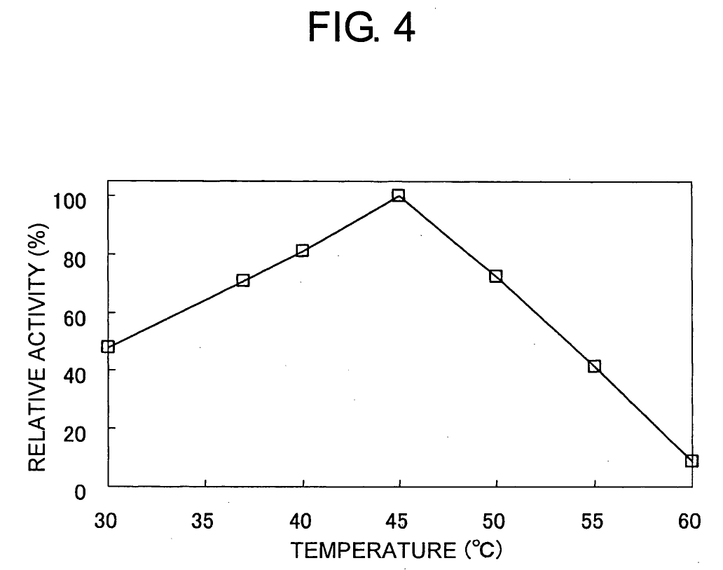

The (2S,3S)-2,3-butanediol dehydrogenase activity of the enzyme obtained in Example 1 was measured by changing just the temperature of the standard reaction conditions, and the results are shown in FIG. 4. The enzyme activity is expressed as relative activity, taking the maximum activity as 100. The optimal temperature was 45° C.

### Example 6

### Substrate Specificity of (2S,3S)-2,3-butanediol dehydrogenase

The dehydrogenase activity of the enzyme obtained in Example 1 was measured against various substrates (50 mM) under the standard reaction condition. The results are shown in Table 2. The enzyme activity is expressed as relative activity, taking the dehydrogenase activity on (2S,3S)-2,3-butanediol as 100.

TABLE 2

| Substrate | Relative activity (%) |
|---|---|
| (2S,3S)-2,3-butanediol | 100 |
| (2R,3R)-2,3-butanediol | 2.89 |
| meso-2,3-butanediol | 0 |
| Acetoin | 0.26 |
| (S)-2-butanol | 43.3 |
| (R)-2-butanol | 1.11 |
| (S)-1,2-propanediol | 55.3 |
| (R)-1,2-propanediol | 0.90 |
| (S)-3-chloro-1,2-propanediol | 0 |
| (R)-3-chloro-1,2-propanediol | 3.62 |

The reductive activity of the enzyme obtained in Example 1 was measured on various substrates (50 mM) under the standard reaction condition. The results are shown in Table 3. The enzyme activity is expressed as relative activity, taking the reductive activity on 2,3-butanedione as 100.

TABLE 3

| Substrate | Relative activity (%) |
|---|---|
| 2,3-Butanedione | 100 |
| Acetoin | 58.9 |

### Example 7

### Measurement on activation of (2S,3S)-2,3-butanediol dehydrogenase

The (2S,3S)-2,3-butanediol dehydrogenase activity was measured under the standard reaction condition in the presence of various divalent ions (1 mM), and the results are shown in Table 4. The enzyme activity is expressed as relative activity, taking the (2S,3S)-2,3-butanediol dehydrogenase activity in the absence of the ions as 100. The (2S,3S)-2,3-butanediol dehydrogenase of thepresent invention was hardly activated by these divalent ions.

TABLE 4

| Added ion | Relative activity (%) |
|---|---|
| None | 100 |
| Mg²⁺ | 110 |
| Ca²⁺ | 113 |
| Ba²⁺ | 112 |
| Mn²⁺ | 104 |
| Co²⁺ | 106 |
| Ni²⁺ | 104 |
| Zn²⁺ | 109 |
| Cu²⁺ | 107 |

### Example 8

### Partial Amino Acid Sequence of the (2S,3S)-2,3-butanediol dehydrogenase

The enzyme obtained in Example 1 was used to determine the N-terminal amino acid sequence with protein sequencer. The amino acid sequence is shown in SEQ ID NO: 3. Furthermore, a fragment containing the (2S,3S)-2,3-butanediol dehydrogenase was cut out from the SDS-PAGE gel, and after washing twice, in-gel digestion was carried out overnight at 35° C. using lysyl endopeptidase. The digested peptide was separated and collected by a gradient elution with acetonitrile in 0.1% trifluoroacetic acid (TFA) using reverse phase HPLC (Tosoh TSK Gel ODS-80-Ts, 2.0 mm×250 mm). The amino acid sequences of the collected peptide fragments were analyzed with protein sequencer (Applied Biosystems). One amino acid sequence was obtained as the N-terminal amino acid sequence. Furthermore, the amino acid sequence of peptide A is shown in SEQ ID NO: 4.

SEQ ID NO: 3: N-terminal amino acid sequence
Met-Ser-Leu-Asn-Gly-Lys-Val-Ile-Leu-Val-Thr
SEQ ID NO: 4: Peptide A
Ile-Ile-Asn-Ala-Cys-Ser-Ile-Ala-Gly-His

### Example 9

### Preparation of Chromosomal DNA from Zoogloea ramigera

The chromosomal DNA of Zoogloea ramigera strain DSM 287 was purified by the Cetyltrimethylammonium bromide (CTAB) method (Current Protocol, Unit 2.4 Preparation of Genomic DNA from Bacteria).

### Example 10

### Cloning of the Core Region of the (2S,3S)-2,3-butanediol dehydrogenase gene by PCR

Sense primer N corresponding to the N-terminal amino acid sequence, and antisense primer A and B corresponding to Peptide A were synthesized. The respective nucleotide sequences are shown in SEQ ID NOs: 5 (primer N), 6 (primer A) and 7 (primer B).

Primer N: (SEQ ID NO: 5)
GTCGAATTCAAYGGCAARGTSATYYTSGTNAC
Primer A: (SEQ ID NO: 6)
GTCGAATTCGCRATSGARCASGCRTTRATDA
Primer B: (SEQ ID NO: 7)
GTCGAATTCGCRATRCTRCASGCRTTRATDA

### Example 11

### PCR Conditions

A 50 μL reaction solution containing 250 ng chromosomal DNA derived from Zoogloea ramigera, 2.0 U ExTaq, 20 pmol primer N, 20 pmol primer mixture containing equal amounts of primers A and B, 20 nmol dNTPs and ExTaq buffer was applied to GeneAmp PCR System 2400 (Applied Biosystems), heated at 94° C. for 2 minutes and 30 seconds, and then 30 cycles of 94° C. for 30 seconds, 45° C. for 30 seconds and 70° C. for 1 minute was performed. As a result, a specific band was obtained.

### Example 12

### Subcloning of PCR Fragment From the Core Region of the (2S;3S)-2,3-butanediol dehydrogenase gene

The DNA fragment obtained in Example 11 was purified by electrophoresis using agarose. The purified DNA fragment was digested with the restriction enzyme EcoRI and ligated with vector pUC118 EcoRI/BAP (TaKaRa) using the Takara Ligation Kit. E. coli JM109 strain was transformed with the resulting DNA construct, and were grown on a plate with LB medium (1% bacto-tryptone, 0.5% bacto-yeast extract and 1% sodium chloride; hereinafter abbreviated as LB medium) containing ampicillin (50 μg/ml).

The plasmid of interest was purified from the transformed strain comprising it, and then the nucleotide sequence of the inserted DNA was analyzed. PCR was performed with Big-Dye Terminator Cycle Sequencing ready Reaction Kit (Applied Biosystems), and then the nucleotide sequence of the DNA was analyzed on PRISM 377 DNA Sequencer (Applied Biosystems). The determined nucleotide sequence of the core region is shown in SEQ ID NO: 8.

### Example 13

### Subcloning of DNA Regions Adjacent to the Core Region of the (2S,3S)-2,3-butanediol dehydrogenase gene

The Zoogloea ramigera-derived chromosomal DNA was digested with each of the restriction enzymes BstYI, NspI, EcoRI, BamHI, PstI and XbaI, and then self-ligated at 16° C. overnight using T4 ligase to cyclize each fragment. Subsequently, PCR was performed in 50 μL reaction mixture containing primers ZrDH-c5u (100 pmol; SEQ ID NO: 9) and ZrDH-c3d (100 pmol; SEQ ID NO: 10), dNTPs (10 nmol), the self-ligated DNA (100 ng), LA-Taq buffer (TaKaRa) and LA-Taq (2.0 U) (TaKaRa) with 30 cycles of denaturation (94° C. for 30 seconds), annealing (60° C. for 30 seconds) and extension (72° C. for 10 minutes) on GeneAmp PCR System 2400 (Applied Biosystems). Aliquots of the PCR products were analyzed by agarose gel electrophoresis, and the result detected DNA fragments of about 2800 bp and 3600 bp corresponding to the template DNA digested with BstYI and BamHI, respectively. The obtained DNA fragments were dubbed ZrDH-1 and ZrDH-2:

ZrDH-c5u (SEQ ID NO: 9)
GCGAGATCAGCGCCTTCC;
and
ZrDH-c3d (SEQ ID NO: 10)
TGTCGACGGCGTGCTCTG.

Each of the PCR-amplified DNA fragments was purified by electrophoresis using agarose. PCR was performed on the purified DNA fragments using Dye Terminator Cycle Sequencing FS ready Reaction Kit (Applied Biosystems), and PRISM 377 DNA Sequencer (Applied Biosystems) was used for sequencing.

The nucleotide sequence of each of the analyzed DNA fragments was divided into the 5′-upstream side (5U) and the 3′-downstream side of the core region, and are shown as ZrDH-5U (SEQ ID NO: 11) and ZrDH-3D (SEQ ID NO: 12), respectively.

The sequence of the (2S,3S)-2,3-butanediol dehydrogenase gene was determined by open reading frame (ORF) search using the nucleotide sequences of ZrDH-1 and ZrDH-2. The determined DNA sequence is shown in SEQ ID NO: 1, and the protein sequence encoded thereby in SEQ ID NO: 2. The alignment and ORF search were performed by Genetyx-ATSQ/WIN and Genetyx-WIN programs (both are from Software Development Co.).

### Example 14

### Cloning of the (2S,3S)-2,3-butanediol dehydrogenase gene

Primers ZrDH-AT1 (SEQ ID NO: 13) and ZrDH-TA1 (SEQ ID NO: 14) for the construction of expression vector were synthesized based on the nucleotide sequence of the structural gene of the (2S,3S)-2,3-butanediol dehydrogenase. PCR was carried out using 50 μL reaction mixture containing each of the primers (20 pmol each), dNTPs (20 nmol), Zoogloea ramigera-derived chromosomal DNA (100 ng), Pfu-DNA polymerase buffer (STRATAGENE) and Pfu-DNA polymerase (2.5 U; STRATAGENE) with 30 cycles of denaturation (95° C. for 30 seconds), annealing (55° C. for 1 minute) and extension (72° C. for 1 minutes and 30 seconds) on GeneAmp PCR System 2400 (Applied Biosystems).

ZrDH-AT1 (SEQ ID NO: 13):
GTCGAATTCAATCATGAGTTTAAATGGCAAAGTCATTTTGGTAACC;
and
ZrDH-TA1 (SEQ ID NO: 14):
GTCAAGCTTCTAGATTAACGATAGACGATACCGCCATC.

As a result of analyzing a part of the PCR product by agarose gel electrophoresis, a specific band was detected.

The obtained DNA fragment was purified by electrophoresis on 1% low-melting point agarose. The purified DNA fragment was double-digested with restriction enzymes EcoRI and HindIII, and agarose gel electrophoresis was performed. The band of interest was cut out and then purified using Sephaglas (Amersham Biosciences).

The resulting DNA fragment was ligated with EcoRI-HindIII double-digested pSE42oD (JP-A2000-189170) using Takara Ligation Kit, and E. coli JM109 strain was transformed with this construct.

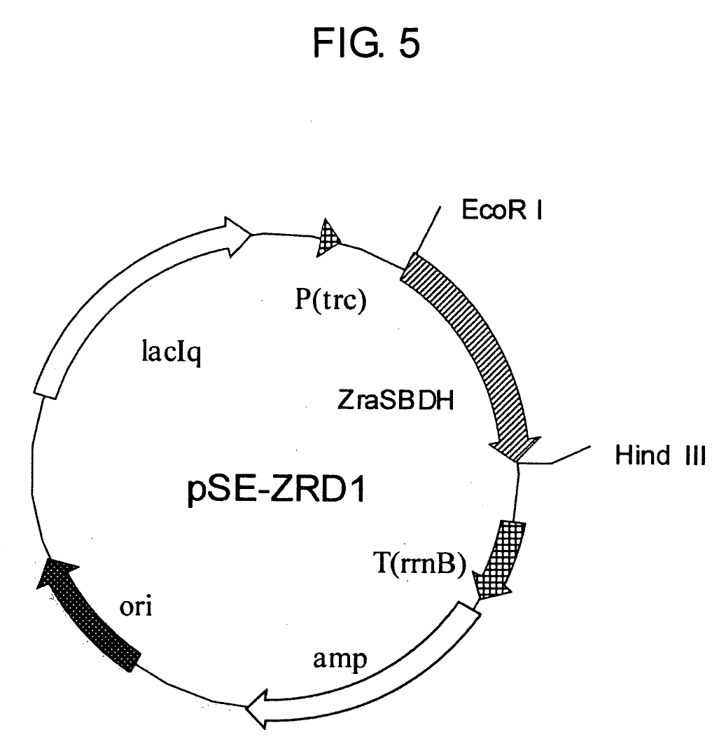

The transformed strain was grown on an LB medium plate containing ampicillin (50 μg/ml), andplasmids were purified from some of the colonies. Then, the nucleotide sequences of inserted fragments were analyzed. A plasmid of interest, which contained the (2S,3S)-2,3-butanediol dehydrogenase gene, was dubbed pSE-ZRD1. The map of the constructed plasmid is shown in FIG. 5.

### Example 15

### Production of Recombinant (2S,3S)-2,3-butanediol dehydrogenase in E. coli

E. coli JM109 strain transformed with the expression plasmid pSE-ZRD1 for the (2S,3S)-2,3-butanediol dehydrogenase gene was cultured in liquid LB medium containing ampicillin at 30° C. overnight, and then 0.1 mM isopropylthiogalactoside (IPTG) was added thereto. The cultivation was further continued for 4 hours.

The bacterial cells were collected by centrifugation and then suspended in 100 mM potassium phosphate buffer (pH 8.0) containing 0. 02% 2-mercaptoethanol. The bacterial cells were disrupted by the treatment with closed sonic chamber device UCD-200TM (Cosmo Bio) for 4 minutes. The solution of disrupted bacterial cells was separated by centrifugation and the resulting supernatant was recovered as a bacterial cell extract.

### Example 16

### Measuring the Activity of the Recombinant (2S,3S)-2,3-butanediol dehydrogenase

The activity on various substrates was measured using the recombinant (2S,3S)-2,3-butanediol dehydrogenase prepared in Example 15. The activity was compared to that of the cell-free extract prepared similarly as in Example 15 from cells without the plasmid. The result of oxidation reaction on (2S,3S)-2,3-butanediol is shown in Table 5 and that of reduction reaction on 2,3-butanedione is shown in Table 6.

TABLE 5

| | (2S,3S)-2,3-butanediol oxidation activity |
|---|---|
| Plasmid | (U/mg-protein) |
| None | 0.0 |
| pSE-ZRD1 | 28.2 |

TABLE 6

| | 2,3-butanedione reduction activity |
|---|---|
| Plasmid | (U/mg-protein) |
| None | 0.0 |
| pSE-ZRD1 | 17.2 |

### Example 17

### Construction of pSF-ZRD1 Plasmid that Coexpresses (2S,3S)-2,3-butanediol dehydrogenase and mycobacterium-derived formic acid dehydrogenase

The pSE-ZRD1 plasmid constructed in Example 14 was double-digested with two restriction enzymes, EcoRI and HindIII, to prepare a DNA fragment comprising the (2S,3S)-2,3-butanediol dehydrogenase enzyme gene derived from Zoogloea ramigera.

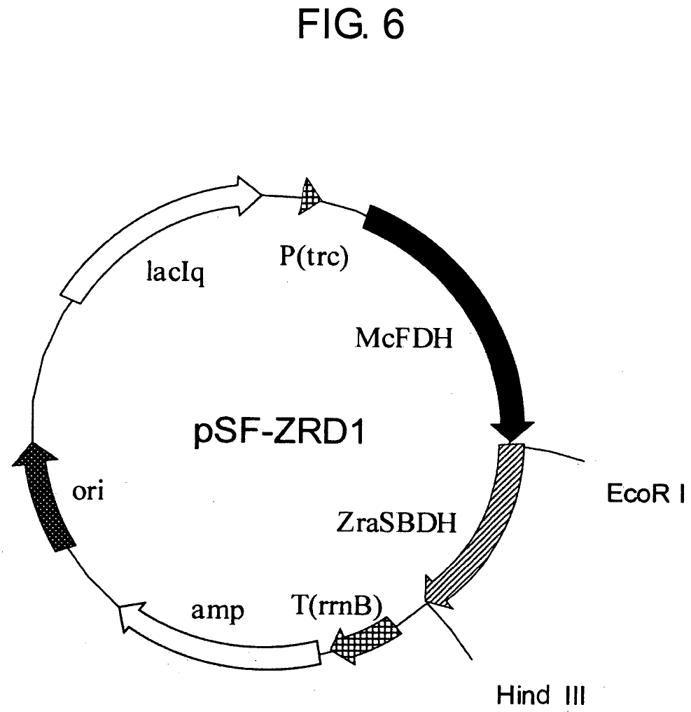

pSE-MF26 plasmid that expresses the mycobacterium-derived formic acid dehydrogenase (Japanese Patent Application No. 2002-207507) were double-digested with two restriction enzymes, EcoRI and HindIII, to prepare a DNA fragment comprising the mycobacterium-derived formic acid dehydrogenase gene. The DNA fragment was ligated using T4 DNA ligase to the DNA fragment comprising the Zoogloea ramigera-derived (2S,3S)-2,3-butanediol dehydrogenase gene, which was cut out from pSE-ZRD1 using the same enzymes, to obtain pSF-ZRD1 plasmid that can simultaneously express both the formic acid dehydrogenase and the (2S,3S)-2,3-butanediol dehydrogenase. The map of the constructed pSF-ZRD1 plasmid is shown in FIG. 6.

### Example 18

### Simultaneous Expression of (2S,3S)-2,3-butanediol dehydrogenase and mycobacterium-derived formic acid dehydrogenase in E. coli

E. coli strain JM109 was transformed with the pSF-ZRD1 plasmid that can coexpress the mycobacterium-derived formic acid dehydrogenase and the Zoogloea ramigera-derived (2S,3S)-2,3-butanediol dehydrogenase.

The recombinant E. coli cells were seeded in LB liquid medium, cultured overnight at 30° C., and IPTG (0.1 mM) was added to further culture the cells for another 4 hours. The obtained E. coli cells were collected and enzyme activity was measured.

### Example 19

Enzyme activity of E. coli transformed with pSF-ZRD1 E. coli cells transformed with pSF-ZRD1 prepared in Example 18 (corresponding to 6.3 mL of the culture solution) were disrupted according to the method of Example 15 to prepare bacterial extract solution (crude enzyme solution) . The obtained extract was used to measure enzyme activity. Measurement of formic acid dehydrogenase activity was performed at 30° C. in reaction solution containing 100 mM potassium phosphate buffer (pH 7.0), 2.5 mM NAD⁺, 100 mM formic acid and the enzyme. 1 U was defined as the amount of enzyme that catalyzes the production of 1 pmol NADH in 1 minute under the above-mentioned reaction condition. The enzyme activity of the crude enzyme solution obtained from the recombinant E. coli was 17.4 U/mg-protein for the (2S,3S)-2,3-butanediol dehydrogenase activity and 0.105 U/mg-protein for the formic acid dehydrogenase activity.

While the invention has been described in detail and with reference to specific embodiments thereof, it will be apparent to one skilled in the art that various changes and modifications can be made therein without departing from the spirit and scope of the invention.

1. A method of producing an alcohol, wherein the method comprises the steps of:
(a) reacting an enzymatically active substance selected from the group consisting of a (2S,3S)-2,3-butanediol dehydrogenase, a microorganism producing such an enzyme, and treated products thereof, with a ketone in the presence of the reduced form of nicotinamide adenine dinucleotide, wherein the (2S,3S)-2,3-butanediol dehydrogenase has the following physicochemical properties (1) to (3):
(1) Function:
produces (S)-acetoin by acting on (2S,3S)-2,3-butanediol using nicotinamide adenine dinucleotide as a coenzyme, and reduces 2,3-butanedione using the reduced form of nicotinamide adenine dinucleotide as a coenzyme to produce (2S,3S)-2,3-butanediol;
(2) Substrate specificity:
uses nicotinamide adenine as a coenzyme for oxidation reaction, utilizes the reduced form of nicotinamide adenine dinucleotide as a coenzyme for reduction reaction, and preferentially oxidizes (2S,3S)-2,3-butanediol among the three isomers of 2,3-butanediol;
(3) Activation by divalent ions:
substantially not activated by Mg²⁺, Ca²⁺, Ba²⁺, Co²⁺ or Mn²⁺ ion.;
and
(b) collecting the produced alcohol.

2. The method of producing an alcohol of claim 1, wherein the ketone is 2,3-butanedione and the alcohol is (2S,3S)-2,3-butanediol.

3. The method of producing an alcohol of claim 1, wherein the (2S,3S)-2,3-butanediol dehydrogenase further has the following substrate specificity:
(i) preferentially oxidizes the hydroxyl group in (S)-configuration of 2-butanol; and
(ii) preferentially oxidizes the hydroxyl groups in (S)-configuration of 1,2-propanediol.

4. The method of producing an alcohol of claim 1, wherein the (2S,3S)-2,3-butanediol dehydrogenase is produced by a microorganism belonging to the genus Zoogloea.

5. The method of producing an alcohol of claim 4, wherein the microorganism is Zoogloea ramigera.

6. The method of producing an alcohol of claim 1, wherein the (2S,3S)-2,3-butanediol dehydrogenase is encoded by a polynucleotide selected from the group of:
(a) a polynucleotide comprising the nucleotide sequence of SEQ ID NO: 1;
(b) a polynucleotide encoding a protein comprising the amino acid sequence of SEQ ID NO: 2;
(c) a polynucleotide encoding a protein comprising the amino acid sequence of SEQ ID NO: 2, wherein one or more amino acids have been substituted, deleted, inserted and/or added, further wherein the protein is functionally equivalent to the protein defined in part(b);
(d) a polynucleotide that hybridizes under stringent conditions with a DNA comprising the nucleotide sequence of SEQ ID NO: 1, and encodes a protein which is functionally equivalent to the protein defined in part(b); and
(e) a polynucleotide comprising a nucleotide sequence that has 70% or higher homology to the nucleotide sequence of SEQ ID NO: 1.

7. The method of producing an alcohol of claim 6, wherein the polynucleotide encodes a protein comprising the amino acid sequence of SEQ ID NO: 2, wherein up to 5% of the amino acids have been substituted, deleted, inserted and/or added, further wherein the protein is functionally equivalent to the protein comprising the amino acid sequence of SEQ ID NO: 2.

8. The method of producing an alcohol of claim 6, wherein the polynucleotide strictly hybridizes under highly stringent conditions with a DNA comprising the nucleotide sequence of SEQ ID NO: 1, and encodes a protein which is functionally equivalent to the protein defined in part (b); polynucleotide of part (d) strictly hybridizes under highly stringent conditions with a DNA comprising the nucleotide sequence of SEQ ID NO: 1.

9. The method of producing an alcohol of claim 6, wherein the polynucleotide comprises a nucleotide sequence that has 95% or-higher homology to the nucleotide sequence of SEQ ID NO: 1.

10. The method of producing an alcohol of claim 6, wherein the polynucleotide is contained within a vector.

11. The method of producing an alcohol of claim 7, wherein the vector is carried by a transformant.

12. A method of producing an optically active alcohol, wherein the method comprises the steps of:
(a) reacting an enzymatically active substance selected from the group consisting of a (2S,3S)-2,3-butanediol dehydrogenase, a microorganism producing such an enzyme, and treated products thereof, with a ketone in the presence of the reduced form of nicotinamide adenine dinucleotide, wherein the (2S,3S)-2,3-butanediol dehydrogenase has the following physicochemical properties (1) to (3):
(1) Function:
produces (S)-acetoinbyactingon (2S,3S)-2,3-butanediol using nicotinamide adenine dinucleotide as a coenzyme, and reduces 2,3-butanedione using the reduced form of nicotinamide adenine dinucleotide as a coenzyme to produce (2S,3S)-2,3-butanediol;
(2) Substrate specificity:
uses nicotinamide adenine as a coenzyme for oxidation reaction, utilizes the reduced form of nicotinamide adenine dinucleotide as a coenzyme for reduction reaction, and preferentially oxidizes (2S,3S)-2,3-butanediol among the three isomers of 2,3-butanediol;
(3) Activation by divalent ions:
substantially not activated by Mg²⁺, Ca²⁺, Ba²⁺, Co²⁺ or Mn²⁺ ion;
(b) preferentially oxidizing one of the optical isomers; and
(c) obtaining the remaining optically active alcohol.

13. The method of producing an alcohol of claim 12, wherein the ketone is 2,3-butanedione and the alcohol is (2S,3S)-2,3-butanediol.

14. The method of producing an alcohol of claim 12, wherein the (2S,3S)-2,3-butanediol dehydrogenase further has the following substrate specificity:
(i) preferentially oxidizes the hydroxyl group in (S)-configuration of 2-butanol; and
(ii) preferentially oxidizes the hydroxyl groups in (S)-configuration of 1,2-propanediol.

15. The method of producing an alcohol of claim 12, wherein the (2S,3S)-2,3-butanediol dehydrogenase is produced by a microorganism belonging to the genus Zoogloea.

16. The method of producing an alcohol of claim 15, wherein the microorganism is Zoogloea ramigera.

17. The method of producing an alcohol of claim 12, wherein the (2S,3S)-2,3-butanediol dehydrogenase is encoded by a polynucleotide selected from the group of:
(a) a polynucleotide comprising the nucleotide sequence of SEQ ID NO: 1;
(b) a polynucleotide encoding a protein comprising the amino acid sequence of SEQ ID NO: 2;
(c) a polynucleotide encoding a protein comprising the amino acid sequence of SEQ ID NO: 2, wherein one or more amino acids have been substituted, deleted, inserted and/or added, further wherein the protein is functionally equivalent to the protein defined in part(b);
(d) a polynucleotide that hybridizes under stringent conditions with a DNA comprising the nucleotide sequence of SEQ ID NO: 1, and encodes a protein which is functionally equivalent to the protein defined in part(b); and
(e) a polynucleotide comprising a nucleotide sequence that has 70% or higher homology to the nucleotide sequence of SEQ ID NO: 1.

18. The method of producing an alcohol of claim 17, wherein the polynucleotide encodes a protein comprising the amino acid sequence of SEQ ID NO: 2, wherein up to 5% of the amino acids have been substituted, deleted, inserted and/or added, further wherein the protein is functionally equivalent to the protein comprising the amino acid sequence of SEQ ID NO: 2.

19. The method of producing an alcohol of claim 17, wherein the polynucleotide strictly hybridizes under highly stringent conditions with a DNA comprising the nucleotide sequence of SEQ ID NO: 1, and encodes a protein which is functionally equivalent to the protein defined in part (b); polynucleotide of part (d) strictly hybridizes under highly stringent conditions with a DNA comprising the nucleotide sequence of SEQ ID NO: 1.

20. The method of producing an alcohol of claim 17, wherein the polynucleotide comprises a nucleotide sequence that has 95% or higher homology to the nucleotide sequence of SEQ ID NO: 1.

21. The method of producing an alcohol of claim 17, wherein the polynucleotide is contained within a vector.

22. The method of producing an alcohol of claim 21, wherein the vector is carried by a transformant.
